(19) United States
(12) Patent Application Publication
(10) Pub. No.: US 2003/0130221 A1
(43) Pub. Date: Jul. 10, 2003

(54) Induction of tolerance to a therapeutic polypeptide

(75) Inventors: Katherine A. High, Merion, PA (US); Roland W. Herzog, Glenolden, PA (US); Valder R. Arruda, Philadelphia, PA (US)

Correspondence Address: FOLEY AND LARDNER, SUITE 500, 3000 K STREET NW, WASHINGTON, DC, 20007, US

(73) Assignee: Children's Hospital of Philadelphia

(21) Appl. No.: 10/287,790

(22) Filed: Nov. 5, 2002

Related U.S. Application Data
(60) Provisional application No. 60/331,074, filed on Nov. 7, 2001.

(51) Int. Cl. A61K 48/00; C12N 15/861; C12N 15/867

(52) U.S. Cl. 514/44; 424/93.2; 435/456; 435/320.1

(57) ABSTRACT

Liver-directed gene transfer can induce immunological tolerance to a polypeptide associated with the expression of a therapeutic nucleic acid. Hepatic expression of a transgene induces tolerance to the expression product of the transgene, or to post-translational product related to transgene expression, thereby ameliorating or eliminating the immune responses associated with gene therapy and protein replacement, respectively, independent of the genetic background of the subject.

This application claims priority to U.S. provisional application No. 60/331,074, filed Nov. 7, 2001, the contents of all of which are incorporated herein by reference in their entirety.

Pursuant to 35 U.S.C. §202 (c), it is acknowledged that the U.S. Government has certain rights in the invention described herein, which was made in part with funds from the National Institutes of Health NHLBI Agency, Grant Number HL61921.

### BACKGROUND OF THE INVENTION

1. FIELD OF THE INVENTION

The present invention relates to a gene therapy strategy in which a nucleic acid is expressed in hepatocytes to induce tolerance to a therapeutic protein.

2. DESCRIPTION OF RELATED ART

There is a growing field of medicine that entails the introduction into cells of nucleic acid molecules that, upon transcription and/or translation, function to ameliorate or otherwise treat a disease or modify a trait associated with a particular cell type, tissue, or organ of a subject. For purposes of the present description, these molecules are categorized as “therapeutic nucleic acid molecules.”

Thus, transcription or translation of a given therapeutic nucleic acid molecule may be useful in treating cancer or an acquired disease, such as AIDS, pneumonia, emphysema, or in correcting inborn errors of metabolism, such as cystic fibrosis. Allergen-mediated and infectious agent-mediated inflammatory disorders also can be countered by administering, via the present invention, a therapeutic nucleic acid molecule that, upon expression in a patient, affects immune response(s) associated with the allergen and infectious agent, respectively. A therapeutic nucleic acid molecule also may have an expression product, or there may be a downstream product of post-translational modification of the expression product, that reduces the immunologic sequalae related to transplantation or that helps facilitate tissue growth and regeneration. Alternatively, the expression product or a related, post-translational agent may be a protein, typified by such proteins as α-, β- and δ-globin, insulin, erythropoietin, and TGF-β, to name a few.

In other words, expression of a therapeutic nucleic acid molecule by a host cell can supply a needed compound, mediate a targeted immune response, or interrupt a pathological process. For all of these and other diverse uses of a therapeutic nucleic acid molecule, the present description employs the rubric of “gene therapy,” in relation to methodology or systems for transferring a therapeutic nucleic acid molecule into host cells, not only in vivo but also ex vivo, as described, for instance, in U.S. Pat. No. 5,399,346.

Gene therapy is complicated by the risk of an immune response to the transgene product. Such an immune response is influenced by the transfer vector itself, the target tissue/route of administration, the vector dose administered, levels of transgene expression, and the underlying mutation in the gene defect, e.g., missense versus gene deletion.

Using factor IX (F.IX) deficiency as a model, scientists have been able to dissect the immune response associated with conventional hemophilia treatments, which typically entail protein replacement. Hemophilia is an ideal model for gene therapy because precise regulation and tissue-specific transgene expression is not required. Lozier et al., JAMA 1994, 271:47; High, Circ. Res. 2001, 88:137.

Hemophilia B is a sex-linked bleeding disorder caused by a deficiency of functional coagulation F.IX. Current replacement therapy consists of intravenous (IV) infusion of protein concentrate and clinical endpoints for treatment of hemophilia are well defined. An increase of factor levels to >1% will improve symptoms associated with the disease from severe to moderate, with reduced frequency of spontaneous bleeds, while an increase to >5% would likely require patients to undergo factor infusion only following severe injury or during surgery.

Replacement therapy generally is used after bleeds have occurred, and so chronic joint damage and the risk of a fatal bleed is always present. Additionally, replacement therapy carries the risk of transmitting blood-borne diseases and formation of inhibitory antibodies to the deficient protein. Formation of inhibitory antibodies is the most serious complication and occurs mostly in patients with extensive loss of F.IX coding information. Lee CA (ed.) 1996, CLINICAL HAEMATOLOGY: HAEMOPHILIA (Bailliere Tindall); Ljung et al., Brit. J. Haematol. 2001, 113:81. It is observed with a frequency of 3-4% in hemophilia B patients. Aledort et al. (eds.) 1995, ADVANCES IN EXPERIMENTAL MEDICINE AND BIOLOGY (Plenum Press, NY); Lee Calif., 1996. Induction of tolerance in these so-called inhibitor patients can be achieved by frequent intravenous injections of high doses of clotting factor concentrate in combination with infusion of IgG and immunosuppression. This treatment is inconvenient and expensive, however, costing as much as $1,000,000 per year.

Accordingly, efforts have been made to advance current treatment regimens for hemophilia. Of paramount importance has been identifying a suitable method for factor IX delivery without clinically significant inhibitor antibody formation.

Data from animal studies indicate that inhibitor formation is a frequent complication that can be observed in hemophilia B mice with a large F.IX gene deletion and dogs with a F.IX null mutation, following intramuscular administration of an adeno-associated virus (AAV) vector. Herzog et al., Molec. Ther. 2001, 4:192; Fields et al., Molec. Ther. 2001, 4:201. In these studies, muscle-directed gene therapy only was successful when combined with transient immune suppression.

Other published reports of gene transfer and long-term expression of human factor IX (hF.IX), by means of portal vein infusion of an AAV vector have been more successful. Snyder et al., Nature Med. 1999, 5:64; Snyder et al., Nature Genet. 1997, 16:270; Nakai et al., Blood 1998, 91:4600; Wang et al., Proc. Nat'l Acad. Sci. USA 1999, 96:3906. Antibodies generated against the F.IX transgene product did not significantly affect systemic expression or activity of expressed F.IX. These experiments, however, were only carried out in hemostatically normal or hemophilic C57BL/6 mice. Similar results pertained in hemophilic F.IX deficient C57BL/6 mice after F.IX gene transfer with an adenovirus (Ad) vector and data from the same experiment, but in a different mouse strain, resulted in an inhibitory antibody response. Kung et al., Blood 1998, 91:784. In another study, adenoviral gene transfer of F.IX, following intravenous administration of ad-hF.IX, induced tolerance to the human F.IX antigen, but this, too, occurred only in C57BL/6 mice. Fields et al., Gene Ther. 2001, 8:354. When this experiment was carried out in mice not bred on a C57BL/6 background, inhibitory antibodies developed. Id. Taken together, these findings indicated that there is something unique to the C57BL/6 genetic makeup that does not elicit the antibody response to human coagulation factors typically observed in other strains of mice.

Nathwani et al., Blood 2001, 97:1258, report that AAV-mediated F.IX gene transfer to normal C57BL/6 and BALB/c mice resulted in sustained F.IX expression in association with hepatic delivery but not with intramuscular administration. There is no indication or suggestion that immune tolerance to the F.IX transgene can be induced by any means. These mice were not challenged subsequently with F.IX to demonstrate tolerance; nor were other indicia of a tolerance phenomenon evaluated. In fact, Nathwani et al. suggest that what they called “tolerance” may be a phenomenon induced in subjects during normal development, because of a missense mutation, and not in subjects generally, irrespective of type of genetic mutation.

Other studies report that, in dogs that carry a missense mutation, sustained expression of canine F.IX (cF.IX) was achieved by administering an AAV vector either to the liver, through the portal vein, or to skeletal muscle. Snyder et al, 1999; Wang et al, Molec. Ther. 2000, 1:154; Herzog et al., Nature Med. 1999, 5:56. In none of these studies was there any suggestion of immune tolerance, and no animal was challenged subsequently with either the transgene or exogenous F.IX. Another report indicates that inhibitor antibodies were formed in the context of lentiviral transfer of a cF.IX gene to the liver of dogs with a F.IX null mutation. Kaufman, Human Gene Ther. 1999, 10:2091. On the other hand, hemophilia B dogs with a null mutation of the F.IX gene are reported to have expressed cF.IX, after AAV-mediated delivery of F.IX-encoding DNA to hepatocytes, over a period a few months. Roland W. Herzog et al., Sustained Correction of Canine Hemophilia B By Gene Therapy in Context of a Null Mutation, AMERICAN SOCIETY FOR HEMATOLOGY EDUCATION PROGRAM BOOK Abstract No. 3451 (2000). Given the limited period of sustained F.IX expression and the absence of antibody detection, these results, too, did not implicate induction of immune tolerance in two test animals that displayed sustained expression of F.IX (a third did not).

### SUMMARY OF THE INVENTION

A harmful immune response in a recipient who is not tolerant to the expression product of a therapeutic nucleic acid is a profound obstacle to successful treatment in a number of instances, including hemophilia conditions.

Accordingly, the inventors have developed a gene therapy method to sustain expression of a therapeutic polypeptide. Moreover, the method described here can induce immune tolerance to a transgene product in a human without eliciting a medically significant immune response. Thus, subjects can be treated without the inconvenience and expense associated with using immunosuppressants. Additionally, the gene therapy method of the instant invention can be used prophylactically, thereby minimizing the risks associated with various untreated diseases or disorders.

Therefore, the present invention provides a gene therapy method for a human subject, comprising providing a composition having a vector and a polynucleotide encoding a therapeutic polypeptide to which the subject is immunologically competent, and administering the composition to the subject, such that the therapeutic polypeptide is expressed selectively in hepatocytes of the subject, and thereafter the subject fails to generate a medically-significant immune response to the expressed therapeutic polypeptide. It is preferred that the composition is administered intravenously, preferably through the portal vein, mesenteric vein, or hepatic artery, but any mode of administration which can effect liver-specific expression is preferable. For example, the composition can be administered to the splenic capsule. In another embodiment, the gene therapy method provides a composition that further comprises a pharmaceutically suitable excipient.

Additionally, liver-directed expression can be facilitated by choice of promoter. Accordingly, it is preferred that the polynucleotide described herein is operably linked to a liver-specific promoter. Examples of such a promoter is a human al-antitrypsin promoter. Ubiquitous promoters, however, may also be used. Additionally, liver specific expression may be affected by choice of vector. Therefore, the vector of the composition described herein can be viral or non-viral. Specifically, the vector can be a plasmid, an adenovirus vector, an adeno-associated virus vector, herpes simplex virus vector, lentivirus vector and retrovirus vector. Preferably, the vector is an adeno-associated virus vector.

The therapeutic polypeptide can be substantially any polypeptide or protein that can elicit a desired therapeutic effect. Preferably, the therapeutic polypeptide modulates the blood clotting or coagulation cascade. Still preferred, the therapeutic polypeptide is factor IX.

Also contemplated in the instant invention is a gene therapy method for a subject who suffers from hemophilia. In this method, the therapeutic polypeptide preferably modulates the blood coagulation or clotting cascade, factor IX, for example, and the subject preferably suffers from hemophilia B.

Furthermore, the invention considers a gene therapy method that may be used prophylactically, where the composition described herein is administered before the subject has exhibited immune intolerance to a therapeutic polypeptide, specifically factor IX.

The gene therapy methods described herein can be administered with a pharmaceutically suitable excipient and exclusive of using an immunomodulator.

### BRIEF DESCRIPTION OF THE FIGURES

FIG. 1. Graphs of hF.IX or anti-F.IX plasma levels in three different mouse strains after AAV-EFlα-hF.IX administration to the liver are provided in FIG. 1. FIG. 1 A-F is a graph of hF.IX expression (A) C57BL/6 mice, (B) C57BL/6 mice, (C) BALB/c mice, (D) BALB/c mice, (E) C3H mice and (F) C3H mice after AAV-EFlα-hF.IX administration to the liver. FIG. 1G-H is a graph of IgGI anti-F.IX in ng/ml in (G) C57BL/6, (H) BALB/c and (I) C3H mice. FIG. 1 J-L is a graph of IgG2a anti-F.IX in ng/ml in (J) C57BL/6, (K) BALB/c and (L) C3H mice. FIG. 1 M-O is a graph of IgG2b anti-F.IX in ng/ml in (M) C57BL/6, (N) BALB/c and (0) C3H mice. AAV-EFlα-hF.IX was administered at a dosage of 1×10¹¹ vg/animal (C57BL/6 and BALB/c mice) or 5×10¹¹ vg/animal (C3H mice). Plasma levels of hF.IX or anti-F.IX were measured by ELISA or immunocapture assay, respectively, in immune competent mice as a function of time after liver-directed vector administration (AAV-EF1oc-hF.IX vector). Vertical arrows indicate challenge by subcutaneous administration of 2 μg hF.IX formulated in cFA.

FIG. 2. Graphs of hF.IX or anti-F.IX plasma levels in three different mouse strains after ApoE/hAAT-hF.IX vector administration to the liver are provided in FIG. 2. FIG. 2 A-C is a graph of hF.IX expression (A) C57BL/6 mice, (B) BALB/c mice and (C) C3H mice after AAV-EFlα-hF.IX administration to the liver. FIG. 1D-F is a graph of IgG1 anti-F.IX in ng/ml in (D) C57BL/6 (E) BALB/c (F) C3H mice. FIG. 1 G-I is a graph of IgG2a anti-F.IX in ng/ml in (G) C57BL/6 (H) BALB/c (1) C3H mice. FIG. 1 J-L is a graph of IgG2b anti-F.IX in ng/ml in (J) C57BL/6 (K) BALB/c (L) C3H mice. Plasma levels of hF.IX or anti-F.IX were measured by ELISA or immunocapture assay, respectively, in immune competent mice as a function of time after liver-directed vector administration (AAV-ApoE/hAAT-hF.IX vector, 1×10¹¹ vg/animal). Each line represents an individual animal. Vertical arrows indicate challenge by subcutaneous administration of 2 μg hF.IX formulated in cFA.

FIG. 3A-C. Bar graph indicating anti-hF.IX antibody production in (A) C57BL/6, (B) BALB/c and (C) C3H mice challenged with 2 μg hF.IX protein in complete Freund's adjuvant (cFA).

FIG. 4A-D. Graph of plasma levels of IgGI anti-hF.IX in (A) C57BL/6, (B) BALB/c, (C) C3H and (D) CD-1 mice on day 14 after immunological challenge is demonstrated in FIG. 4. Mice were challenged by subcutaneous administration of 2 μg hF.IX formulated in cFA. Mice were either naive or received hepatic gene transfer with AAV-EF1α-hF.IX (EFlα), AAV-ApoE/hAAT-hF.IX (hAAT), or AAV-GFP (GFP) vector 2-3 months earlier at the indicated vector doses. Each bar represents an individual animal.

FIG. 5. Graph of IgA anti-hF.IX plasma levels in CD-1 mice that had received 2 immunological challenges of by subcutaneous administration of 2 μg hF.IX formulated in FA (data are for day 5 after the second challenge). Mice were naive or had received hepatic gene transfer with AAV-EFlα-hF.IX or AAV-ApoE/hAAT-hF.IX vector at the indicated vector doses. Each bar represents an individual animal.

FIG. 6. Bar graph representing lymphocyte proliferation following in vitro re-stimulation with hF.IX protein. Naive or AAV-EFlα-hF.IX treated mice (portal infusion of 1×10¹¹ vg/animal) were boosted twice with hF.IX formulated in adjuvant (1.5 months after gene transfer for vector treated mice for the first challenge with hF.IX/cFA and 1 month later with hF.IX/iFA) and sacrificed on day 5 after the second boost. Total pooled splenocytes and inguinal lymph node cells (n=3/strain) were cultured in the presence or absence (mock) of hF.IX antigen (10 μg/ml media) for 5 days prior to pulse with ³T-thymidine. ³T-thymidine incorporation was measured by scintillation counting. All lymphocyte cultures were set up in quadriplicate. Average counts per minute (cpm) ±90%-confidence interval are shown. Numbers above bars are stimulation indexes (ratio of cpm for antigen vs. mock-stimulated cells).

FIG. 7A-C. Graphs of anti-hF.IX or anti-hF.X in naive C57BL/6 or C57BL/6 mice that received hepatic AAV-hF.IX transfer at day 14 after subcutaneous challenge are exemplified in FIG. 7. Graph of (A) hF.X after subcutaneous injection of hF.IX protein formulated in cFA, and (B) anti-hF.IX and (C) anti-hF.X after subcutaneous challenge with a mix of hF.IX and hF.X (5 μg each). The AAV-ApoE/hAAT-hF.IX vector was administered at a dosage of 5×10¹⁰ vg/animal. Each bar represents average values for 4 mice ±90%-confidence interval.

FIG. 8. Bar graph of plasma levels of IgGI anti-hF.IX on day 14 after immunological challenge of by subcutaneous administration of 2 μg hF.IX formulated in cFA in C57BL/6 mice that had received adoptive transfer of splenocytes from naïve or vector-treated C57BL/6 mice. Each bar is average antibody titer for 4-5 animals ±90%-confidence interval. Adoptive transfer was by tail vein injection 24 hrs prior to challenge. Vector-treated mice had received hepatic gene transfer with 1×10¹¹ vg/animal of AAV-EFlα-hF.IX (EFlα) or AAV-ApoE/hAAT-hF.IX (hAAT) vector 1.5 months prior to the experiment. Total splenocytes (5×10⁷, bars 1-4), CD4+T cells-depleted splenocytes (1.5×10⁷, bar 7) or MACS purified CD4⁺ T cells (bars 5,6) were transferred. * Difference in anti-hF.IX titer between mice that received splenocytes from naïve vs. from AAV-ApoE/hAAT-hF.IX treated animals was statistically different (p <0.01). ** Difference in anti-hF.IX titer between mice that received CD4⁺ cells from naïve vs. from AAV-ApoE/hAAT-hF.IX treated animals was statistically different (p <0.05).

FIG. 9A-I. Graph of mF.IX systemic expression and coagulation time as measured by aPTT, following hepatic gene transfer with AAV-EFlα-mF.IX or AAV-ApoE/hAAT-mF.IX in hemophilia B mice with a large F.IX gene deletion on different mouse strain backgrounds. FIG. 9 A-B is a graph of (A) coagulation time and (B) F.IX expression on a BALB/c strain background following hepatic gene transfer with AAV-EFlα-nF.IX. FIG. 9 C-D is a graph of (C) coagulation time and (D) F.IX expression on a BALB/c strain background following hepatic gene transfer with AAV-ApoE/hAAT-mF.IX. FIG. 9 E-F is a graph of (E) coagulation time and (F) F.IX expression on a CD-1 strain background following hepatic gene transfer with AAV-EFlα-mF.IX. FIG. 9 G-H is a graph of (G) coagulation time and (H) F.IX expression on a CD-1 strain background following hepatic gene transfer with AAV-ApoE/hAAT-mF.IX. FIG. 9 I-J is a graph of (I) coagulation time and (J) F.IX expression on a C3H strain background following hepatic gene transfer with AAV-ApoE/hAAT-mF.IX. The vector was administered at a dosage of 3×10¹¹ vg/animal. Range of aPTT for normal mouse plasma was 25-35 sec, and >60 sec for untreated hemophilia B mice (as indicated by horizontal lines). Each line represents and individual animal. The dotted line in graph A represents the mouse also depicted in Table 3.

FIG. 10A-E. Graph of (A) whole blood clotting time (WBCT), (B) F.IX expression (C) activated partial thromboplastin time (aPTT), (D) cF.IX antigen levels in plasma, (E) cF.IX activity levels (% activity of pooled normal canine plasma) as a function of time after AAV-ApoE-hAAT vector administration in hemophilia B dogs.

FIG. 11. Western blot analysis of anti-cF.IX IgG in hemophilia B dogs after AAV-ApoE-hAAT vector administration.

### DETAILED DESCRIPTION OF PREFERRED EMBODIMENTS

An important aspect of a successful gene therapy would be an understanding of any associated immune response against the therapeutic polypeptide expressed by transformed cells. A desirable outcome in this regard would be induction of tolerance to the therapeutic polypeptide, not only to prevent a neutralizing antibody response but also to advance gene transfer as a tool for developing tolerance to the therapeutic polypeptide per se, even in the context of conventional replacement therapies.

To this end, the inventors have discovered that liver-directed gene transfer can induce immunological tolerance to a polypeptide associated with the expression of a therapeutic nucleic acid. Using hemophilia as a model, the inventors determined that hepatic expression of a transgene induces tolerance to the expression product of the transgene (or to post-translational product related to transgene expression), thereby ameliorating or eliminating the immune responses associated with gene therapy and protein replacement, respectively, independent of the genetic background of the subject.

In this description, “tolerance” connotes a state characterized by the absence of a medically significant immune response, in an immunologically competent subject, to a therapeutic polypeptide. The induction of tolerance does not mean that the immune system of a subject is incapable of generating an immune response against a therapeutic polypeptide, but rather that the subject's immune system is rendered unresponsive to the presence of the therapeutic polypeptide after hepatic gene delivery. In other words, the subject's immune system will not invoke a medically significant immune response. A “medically significant immune response” is one that interferes substantially with expression or activity of the therapeutic polypeptide or that complicates treatment of the disorder or condition that the therapeutic polypeptide is intended to treat. A “therapeutic polypeptide” is a polypeptide or protein that can elicit a desired therapeutic response.

Against the background of an inconclusive, even inconsistent literature on the immunological consequences of heterologous F.IX expression in transformed animals, the inventors unexpectedly have induced immune tolerance, pursuant to the present invention, in strains of immunocompetent mice other than C57BL/6, thereby showing that the effect is not a function of genotype. After liver-directed administration of a vector encoding F.IX, mice of diverse strains were challenged with the F.IX protein and proved tolerant of the transgene product.

In keeping with these results, the inventors have found that liver-directed gene transfer can sustain correction of canine hemophilia B, without exhibiting a medically significant immune response, in instances of a F.IX gene deletion and a missense mutation alike, with F.IX expression persisting for longer than 14 months. While not proof-positive of tolerance, in the sense that the involved animals have not been challenged with the therapeutic protein, the observation of stable circulating levels of biologically active F.IX for this extended period, absent a medically significant neutralizing/inhibitor antibody response to F.IX, comports with the above-mentioned mouse results and further underscores the unexpected tolerance phenomenon, which opens the way to a new treatment paradigm for human patients . Thus, these results are significant because (i) gene transfer in dogs with a null mutation is associated with a high risk of a inhibitor antibody formation, (ii) high levels of expression can be achieved with relatively low vector doses, and (iii) the dog model is recognized for the predictability of generalizing its results to the human context.

The methodology of the present invention can be used prophylactically, to minimize the symptoms or risks associated with various diseases or disorders. Thus, a tolerized subject challenged with a therapeutic nucleic acid (to effect transgenic expression) or a therapeutic polypeptide directly does not exhibit a medically significant neutralizing/inhibitory antibody response and can ideally prevent or ameliorate symptoms associated with a disease or disorder.

Gene Therapy Method

The instant invention contemplates a gene therapy method for a human subject, comprising (A) providing a composition comprised of a vector and a polynucleotide encoding a therapeutic polypeptide to which said subject is immunologically competent and (B) administering the composition to the subject, such that (i) the therapeutic polypeptide is expressed selectively in hepatocytes of said subject and thereafter (ii) the subject fails to generate a medically-significant immune response to the expressed therapeutic polypeptide.

The methodology of the present invention can be used to treat subjects for whom it is desirable to induce immune tolerance to any given therapeutic polypeptide, whether expressed in situ or administered in the manner of a replacement therapy. Accordingly, the invention contemplates induction of immune tolerance in an individual, such as a hemophiliac, who needs treatment for a genetic defect, even before that individual has exhibited an immune response to the pertinent therapeutic polypeptide. Induction of tolerance by hepatic gene therapy can be achieved through a single, one-time procedure.

For example, hemophilic children can be treated prophylactically with periodic F.IX replacement therapy, which decreases the chance of a fatal bleed due to injury. In addition to the expense and inconvenience of such treatment, repeated F.IX administration results in inhibitor antibody formation in some patients. If the antibodies in these patients are low titer antibodies, patients are treated with larger doses of blood coagulation factors. If the antibodies are high titer antibodies, treatment regimens for these patients become much more complex and expensive. Prophylactic F.IX gene transfer to the liver would induce tolerance to F.IX and allay the problem of inhibitor antibody formation.

Similarly, patients that do not generate neutralizing/inhibitor antibodies in response to protein replacement therapy would also benefit from hepatic delivery of F.IX transgene. Induction of tolerance to the therapeutic protein would result in fewer, if any, F.IX for correction of hemophilia.

Additionally, imrnunosuppression strategies are sometimes coupled with traditional protein replacement therapy to help combat this immune response to the therapeutic polypeptide. Because one can induce antigen-specific immune tolerance by liver directed gene transfer, and therefore reduce or eliminate the risk of an inhibitory antibody response, the present invention preferably comprises administering a composition exclusive of an agent that may modify an immune response, i.e., an immunomodulator.

The Composition

The composition to be administered in the gene therapy method, according to the present invention, comprises a vector and polynucleotide. The polynucleotide encodes a therapeutic polypeptide to which the subject is immunologically competent, i.e., capable of eliciting an immune response. A therapeutic polypeptide as described herein can be a biologically active peptide, protein fragment or full-length protein that can bring forth a desired therapeutic response.

Polynucleotide

The polynucleotide of the present invention can be substantially any nucleic acid that encodes the desired therapeutic polypeptide. The length of the nucleic acid is not critical to the invention, but needs to be of sufficient length to encode a molecule that can exhibit a biological effect. Any number of base pairs up to the full-length gene may be transfected. For example, the polynucleotide may have a length from about 100 to 10,000 base pairs in length, although both longer and shorter nucleic acids can be used.

The polynucleotide can be DNA. For example, linear or circular and can be single- or double-stranded. DNA includes cDNA, triple helical, supercoiled, Z-DNA and other unusual forms of DNA, polynucleotide analogs, antisense DNA, DNA encoding a portion of the genome of an organism, gene fragments, and the like.

The polynucleotide can also be RNA. For example, antisense RNA, viral genome fragments such as viral RNA, RNA encoding a therapeutic protein and the like. The nucleic acid can be selected on the basis of a known, anticipated, or expected biological activity that the nucleic acid will exhibit upon delivery to the interior of a target cell or its nucleus.

Additionally, the polynucleotide may be an autologous or heterologous nucleic acid. A autologous nucleic acid is derived from the same genetic source as the human subject being treated and a heterologous nucleic acid is a nucleic acid derived from a separate genetic source or species. Nucleic acid that is not considered “wild-type” would also be classified as heterologous for purposes of this invention.

The polynucleotide can be prepared or isolated by any conventional means typically used to prepare or isolate nucleic acids. For example, DNA and RNA molecules can be chemically synthesized using commercially available reagents and synthesizers by methods that are described, for example, by Gait, 1985, in OLIGONUCLEOTIDE SYNTHESIS: A PRACTICAL APPROACH (IRL Press, Oxford). RNA molecules also can be produced in high yield via in vitro transcription methods using plasmids such as SP65, which is available from Promega Corporation (Madison, Wis.). The nucleic acid can be purified by any suitable means; many such means are known in the art. For example, the nucleic acid can be purified by reverse-phase or ion exchange HPLC, size exclusion chromatography, or gel electrophoresis. Of course, the skilled artisan will recognize that the method of purification will depend in part on the size of the DNA to be purified. The nucleic acid can also be prepared using any of the innumerable recombinant methods which are known or are hereafter developed.

The polynucleotide encoding one or more proteins of interest can be operatively associated with a variety of different promoter/regulator sequences. The promoter/regulator sequences can include a constitutive or inducible promoter, and can be used under the appropriate conditions to direct high level or regulated expression of the gene of interest. Examples of promoter/regulatory regions suitable for the present invention include a cytomegalovirus (CMV) promoter, elongation factor 1α (EFα) promoter, an α1-antitrypsin promoter and an albumin promoter, but substantially any promoter/regulatory region which preferentially directs high level or regulated expression of the gene to the liver can be used. For example, a synthetic promoter comprised of liver specific promoter and enhancer elements or the ApoE/hAAT enhancer/promoter combination may be used to direct high level expression to the liver. Synthetic promoters are well understood in the field of gene therapy and one skilled in the art would know how to make and use a synthetic promoter suitable for the present invention.

Although preferred, it is not necessary that a liver-specific promoter/regulatory region be used. Gene transfer may be effected to hepatocytes via means other than a liver-specific promoter. For example, vector choice and mode of administration may also influence gene transfer to the liver. It is contemplated in the present invention that any combination of factors may be used to direct transgene expression to the liver.

In a preferred embodiment, the polynucleotide encodes a therapeutic polypeptide that modulates the blood clotting or coagulation cascade. For example, therapeutic polypeptides are preferred that are implicated in the bleeding disorder hemophilia, such as functional blood coagulation factor VIII (hemophilia A) and factor IX (hemophilia B).

Vector

The polynucleotide described here can be recombinantly engineered into a variety of known host vectors that provide for replication of the nucleic acid. These vectors can be designed, using known methods, to contain the elements necessary for directing transcription, translation, or both, of the nucleic acid in a cell to which it is delivered. Known methodology can be used to generate expression constructs the have a protein-coding sequence operably linked with appropriate transcriptional/translational control signals. These methods include in vitro recombinant DNA techniques and synthetic techniques. For example, see Sambrook et al., 1989, MOLECULAR CLONING: A LABORATORY MANUAL, Cold Spring Harbor Laboratory (New York); Ausubel et al., 1997, CURRENT PROTOCOLS IN MOLECULAR BIOLOGY, John Wiley & Sons (New York). Also provided for in this invention is the delivery of a polynucleotide not associated with a vector.

Vectors suitable for use in the instant invention can be viral or non-viral. Particular examples of viral vectors include adenovirus, AAV, herpes simplex virus, lentivirus, and retrovirus vectors. AAV vectors can be produced in a helper virus-free system, are devoid of any viral gene products, and have reduced immunogenicity compared with other viral vectors. Carter et al., Int'l J. Molec. Med. 2000, 6(1):17-27. Therefore, an AAV vector is preferred even though any vector that can help achieve efficient hepatic gene transfer is useable. An example of a non-viral vector is a plasmid.

The vector and polynucleotide described herein may be an expression construct comprising DNA encoding a protein or an expression construct comprising RNA that can be directly translated to generate a protein product. Typically, an expression construct comprises a vector, a promoter/regulatory sequence, a polynucleotide and a polyadenylation signal.

Pharmaceutical Excipient

The composition to be delivered in the gene therapy method described herein can consist of the composition alone in a form suitable for administration to a subject, or can comprise one or more pharmaceutically suitable excipients, one or more additional ingredients, or some combination of these.

Accordingly, another aspect of the present invention is a gene therapy method for a human subject, comprising (A) providing a composition comprised of a vector, a polynucleotide encoding a therapeutic polypeptide to which said subject is immunologically competent and a pharmaceutical excipient and (B) administering said composition to the subject, such that (i) said therapeutic polypeptide is expressed selectively in hepatocytes of the subject and thereafter (ii) said subject fails to generate a medically-significant immune response to the expressed therapeutic polypeptide.

The compounds can be formulated for intravenous administration via, for example, bolus injection or continuous infusion. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that the present composition be introduced into patients via the portal vein, mesenteric vein or hepatic artery.

Additionally, the invention contemplates delivering the composition described herein with a cationic macromolecule or other agent that enhances transfection/infection efficiency. The cationic macromolecule is positively charged, comprises two or more art-recognized modular units (e.g., amino acid residues, fatty acid moieties, or polymer repeating units) and preferably is capable of forming supermolecular structures (e.g., aggregates, liposomes, or micelles) at high concentration in aqueous solution or suspension. Among the types of cationic macromolecules that can be used are cationic lipids, polycationic polypeptides and polymers.

Modes of Administration

The composition of the present invention is designed to achieve selective expression of the therapeutic polypeptide in the hepatocytes of a subject. Liver-directed gene transfer can be accomplished through choice of promoter, choice of vector, or mode of administration, or through a combination of these. Preferably, the composition described herein is administered intravenously, although direct injection into the liver or splenic capsule is also contemplated. Still preferred, liver-directed gene transfer is accomplished by administering the composition through the mesenteric vein, portal vein or portal artery of the subject. Alternatively, the composition may be administered through a peripheral vein of the subject. Thus, any mode of administration that results in sufficient hepatocyte transduction/infection is suitable.

The invention is further described by reference to the following examples, which are provided for illustration only. The invention is not limited to the examples but rather includes all variations that are evident from the teachings provided herein.

### EXAMPLE 1.

Viral Vectors

Example 1.1 AA V-EFlα-hF.IX vector construction

To construct AAV-EFlα-mF.IX, the hF.IX CDNA in AAV-EFlα-hF.IX was replaced with a EcoRI-HindIII fragment that includes the coding sequence of the murine F.IX (mF.IX) cDNA (Fields et al. (2001)). The AAV-EFlα-hF.IX vector for expression of the hF.IX cDNA from the human elongation factor-loc enhancer/promoter (including the first intron of the human EFlα gene) is as described in Nakai et al., Blood (1998) 91:4600.

Example 1.2 AA V-(ApoE)4/hAA T-cF. IX vector construction

Vector AAV-(ApoE)4/hAAT-cF.IX was constructed by replacing the CMV enhancer/promoter in the previously described expression cassette with a liver-specific ApoE/hAAT enhancer/promoter combination. Herzog et al., 1999. This 1.1-kb sequence is comprised of the human α1-antitrypsin promoter and four copies of the ApoE enhancer, as described by Le et al., Blood 1997, 89:1254. The expression cassette also contains a chimeric b-globin/CMV intron, the canine F.IX CDNA, and the human growth hormone polyadenylation (hGH poly A) signal as described. Herzog et al., 1999. AAV2 vector was produced by triple transfection of HEK-293 cells in a helper virus-free system, which utilizes two helper plasmids to supply adenoviral gene functions (E2A, E4, and VA) and the AAV2 rep/cap genes. Matsushita et al., Gene Ther. 1998, 5:938. Plasmids were grown in E.coli DH5a cells and purified using the Qiagen (Santa Clara, Calif.) Giga kit for preparation of endotoxin-free DNA. The AAV helper plasmid has been engineered to increase cap expression and to decrease generation of wild-type AAV to undetectable levels ( <1 in 109 vector particles) in a replication center assay. Matsushita et al. 1999, IMPROVEMENTS IN AAV VECTOR PRODUCTION: ELIMINATION OF PSEUDO-WILD TYPE AAV (WASHINGTON, D.C.). AAV vector was purified from cell lysates by repeated rounds of CsCl density gradient centrifugation, as described by Xiao et al., J. Virol. 1996, 70:8098, and Kay et al., Science 1993, 262:117. Vector was osmotically stabilized in HEPES-buffered saline, pH 7.8, filter-sterilized, and stored at −80° C. prior to use. Vector titers were determined by quantitative slot blot hybridization. The Limulus amoebocyte lysate assay (Sigma, St. Louis, Mo.) was performed to confirm absence of detectable endotoxin in vector preparations.

### EXAMPLE 2

Mouse Strains Experimental Assays

Example 2.1 Mouse Strains

Hemostatically normal C57BL/6, BALB/c, C3H, and CD-1 mice as well as γδ-T cell receptor deficient, CD8⁺ T cell deficient, IL-4 deficient, and Fas-deficient mice (all on a C57BL/6 genetic background) were purchased from Jackson Laboratory. AAV vector (25 μl per injection) was delivered into the portal vein via splenic capsule injection with a Hamilton syringe following a ventral midline incision as described (Nakai et al., Blood (1998) 91:4600 and Mingozzi et al., J. Virol. (2002) 76 in press). For hemostatically normal mice, this procedure could be carried out as published. Hemophilia B mice without endogenous F.IX expression (due to a targeted deletion of the promoter and the first 3 exons of the F.IX gene (Lin et al., Blood (1997) 90:3962) received pooled normal mouse plasma (200 μl) by tail vein injection <30 min prior and after surgery. These mice were generated by repeated breeding of F.IX knock out mice with BALB/c, C3H, or CD-1 mice (at least 10 generations to obtain pure genetic backgrounds). Hemophilia B mice were identified by PCR-based genotyping as described earlier (Fields et al., Mol. Ther. (2001) 4:201. Immunizations were done by subcutaneous injection on the back of 2-5 μg human F.IX or F.X (or 10 μg of murine F.IX in hemophilia B mice) protein formulated in complete or incomplete Freund's adjuvant (cFA or iFA, GIBCO/BRL, Rockville, Md.). Normal mice were bled from the retro-orbital plexus using heparinized capillary tubes, while blood from hemophilia B mice was collected in 0.38%-sodium citrate buffer during bleed from the tail vein (Kung et al. Blood, (1998) 91:784).

Example 2.2 Factor IXand antibody assays

Plasma levels of hF.IX antigen were measured by hF.IX-specific ELISA (Walter et al., Proc. Natl. Acad. Sci. USA (1996), 93:3056). Coagulation of plasma samples obtained from hemophilia B mice was determined by measurement of activated partial thromboplastin time (aPTT) using a fibrometer (Fields (2001). Murine F.IX plasma concentrations were determined by ELISA using purified mF.IX protein as standard (Id.). Briefly, micotiter plates were coated with affinity purified rabbit anti-mF.IX (Zymed Laboratories, South San Francisco, Calif., 1:50 dilution) blocked with non-fat dry milk in 1 x PBS/0.05% Tween-20, and mF.IX was detected with rabbit anti-mF.IX (1:2000 dilution) chemically linked to horseradish peroxidase (conjugation of the antibody was performed with the peroxidase labeling kit from Zymed). Using these reagents, we determined the concentration of normal mouse plasma (C57BL/6-derived) to be 2500 ng/ml, half the normal concentration of hF.IX in human plasma. Immunocapture assay for determination and quantitation of anti-hF.IX or anti-mF.IX immunoglobulin subclasses (IgG1, IgG2a, IgG2b, IgG3, IgA, and IgE) was performed as published using antibodies from Roche Molecular Biochemicals (Indianapolis, Ind.) and immunoglobulin standards from Sigma (St. Louis, Mo.) (Fields (2001) and Fields et al. Mol. Ther. (2000) 1:225). Bethesda assay for measurement of inhibitory anti-mF.IX was as described (Fields (2001)). One Bethesda unit is equivalent to 50% of residual F.IX activity in this plasma-based coagulation assay.

Example 2.3 Cytokine release and T cell proliferation assays

Mice that had received 2 subcutaneous injections with 2 μg of hF.IX in FA (1-1.5 months apart, the first challenge in cFA, the second in iFA) were sacrificed 5 days after the second challenge, and lymphocytes extracted from spleens, portal and inguinal lymph nodes were isolated as described in Fields (2000) and pooled for in vitro T cell assays (3-5 animals/experimental cohort). Lymphocytes were cultured in 2-MLC medium (DMEM, 2% heat-inactivated fetal calf serum, 1 mM sodium pyruvate, 10 mM HEPES, 0.1 mM non-essential amino acids, 10⁻⁶ M 2-mercaptoethanol, and antibiotics) for 5 days in the absence (mock) or presence of hF.IX antigen (10 μg/ml). Source of hF.IX protein for FA immunization and in vitro re-stimulation was high purity plasma-derived hF.IX (Mononine, Armour Pharmaceutical Co., Kankakee, Ill.). Conditioned media were analyzed for cytokine secretion by ELISA as described (Fields (2000)). IL-2 and IFN-γ were measured after 3 days of in vitro re-stimulation, IL-10 and TGF-β after 5 days. ELISA reagents and antibodies were purchased from Pharmingen (San Diego, Calif.) or Promega (Madison, Wis., TGF-β ELISA). [For outbred CD-1 mice, lymphocytes from 3 mice were processed and assayed individually, and cytokine release was averaged.] Lymphocyte proliferation was measured by scintillation count of ³H-thymidine incorporation in hF.IX vs. mock-stimulated cells (48 hrs of in vitro re-stimulation followed by 8 hrs thymidine pulse). Cytokine release and proliferation in response to in vitro re-stimulation with hF.IX antigen was also determined for lymphocytes cultured in the presence of 10, 50, or 100 U/ml of murine IL-2 (Pharmingen).

### EXAMPLE 3

Sustained hF.IX expression following AAV-EFlα-hF.IX or AAV-ApoE/hAAT-hF.IX administration in mice

Example 3.1

Two vectors, AAV-EFlα-hF.IX and AAV-ApoE/hAAT-hF.IX, were produced for expression of hF.IX from the ubiquitous EFlα promoter or a hepatocyte-specific ApoE enhancer/human α1-anti-trypsin promoter combination. These vectors were infused into the portal circulation via the spleen for efficient gene transfer to the liver. Recipients of gene transfer were male immunocompetent mice of three different inbred strains with defined MHC haplotypes: C57BL/6 (H-2^{b}), BALB/c (H-2^{d}), and C3H (H-2^{k}). All strains form neutralizing anti-hF.IX after IM administration of vector (Fields (2000) and unpublished results).

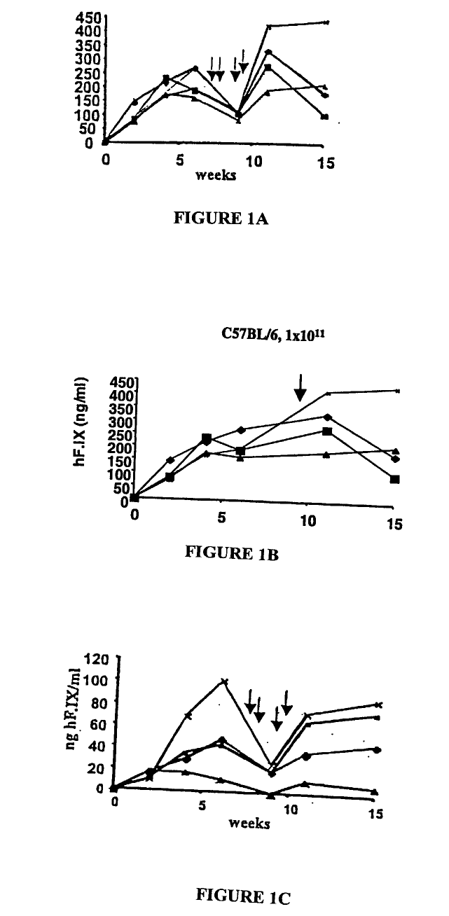

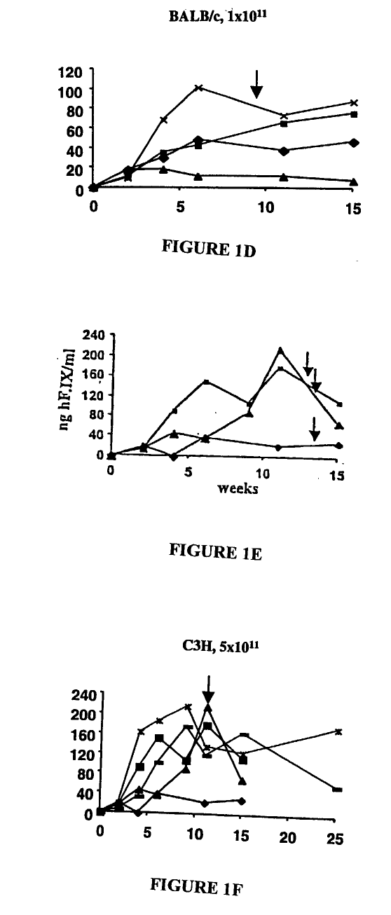

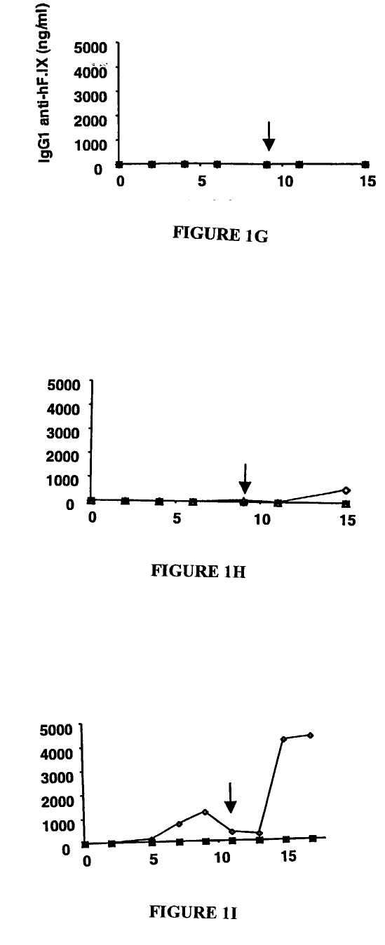

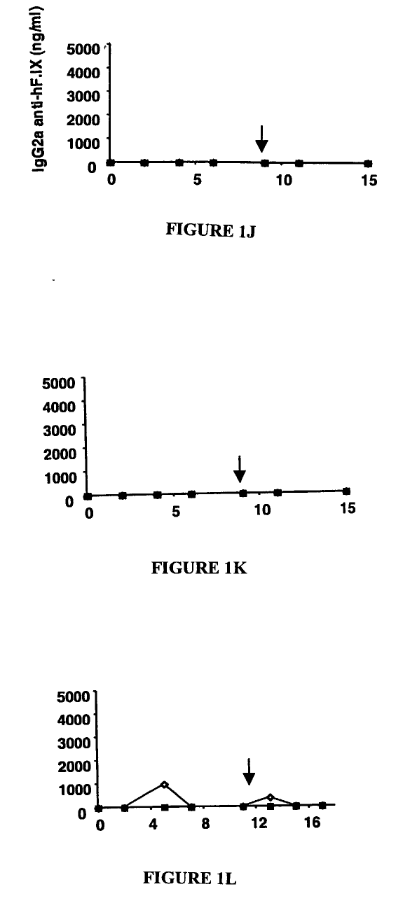

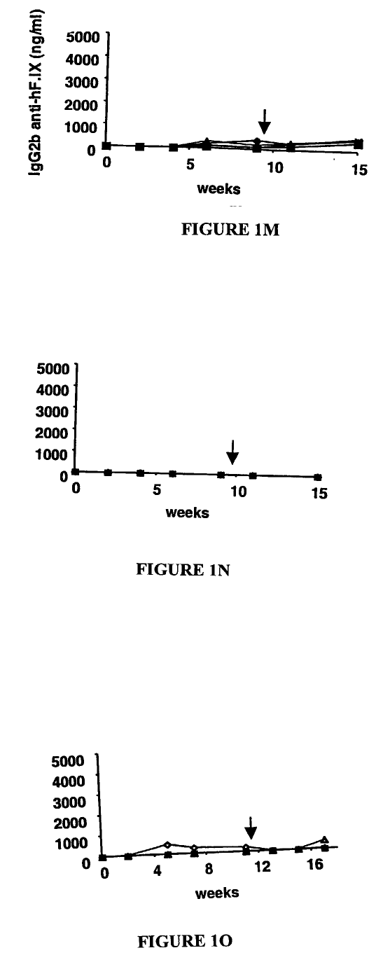

The AAV-EFlα-hF.IX vector was infused into the portal vein of C57BL/6, BALB/c and C3H mice. Strong hF.IX expression was detected by immunofluorescence in the hepatocytes following portal vein injection (FIG. 1A and IC). C57BL/6 mice had the highest levels of expression (100-400 ng/ml), followed by BALB/c mice (50-100 ng/ml) and C3H mice (10 ng/ml). BALB/c mice and C57BL/6 mice continued to express hF.IX for the duration of the experiment (>3 months) without or a weak, non-neutralizing IgG2b anti-hF.IX response, respectively. C3H mice eventually produced anti-hF.IX antibodies at late time points (2.5 months). These 3 mouse strains showed a delayed humoral immune response, if any, against hF.IX in liver-directed gene transfer.

As the data point at 8 weeks in FIGS. 1A and 1C appears to represent an error in the ELISA assay used to measure the F.IX concentrations, the figures have been redrawn without this time point. See FIGS. 1B and 1D. No anti-hF.IX was measured in BALB/c mice (FIG. 1 H, K, N). C57BL/6 developed low titer, non-neutralizing IgG2b at late time points, and C3H mice had no anti-hF.IX (4/5) or an IgG response that was not neutralizing to transgene expression (1/5, FIG. 1I, L, O).

Moreover, 3/3 C3H mice injected with a higher vector dose (5×10¹¹ vg) continue to express 30-200 ng/ml hF.IX (FIG. 1E-3 months, experiment ongoing; 2/3 mice had no anti-hF.IX antibodies; 1/3 mice has a low titer antibody but continues to express hF.IX). The data from this experiment was compiled with additional data (additional animals and time points) and is represented in FIG. 1F.

In an earlier experiment, one C3H mouse and one BALB/c mouse was injected via the portal vein vector-hF.IX which resulted in hF.IX expression without antibody formation (80 and 250 ng/ml, respectively) for the duration the mice were followed.

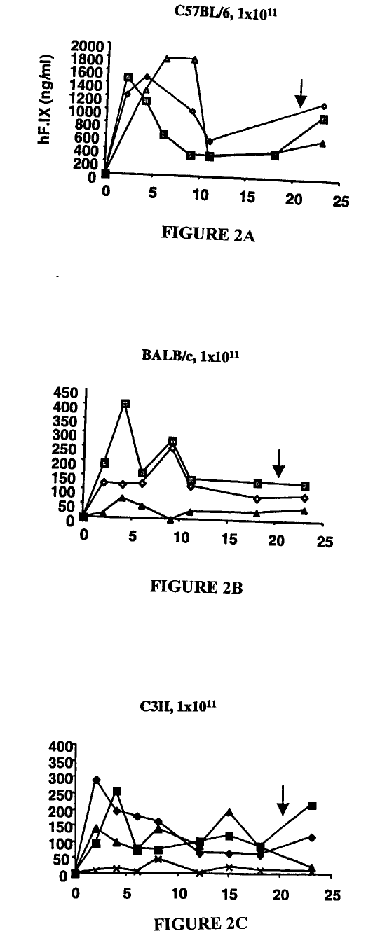

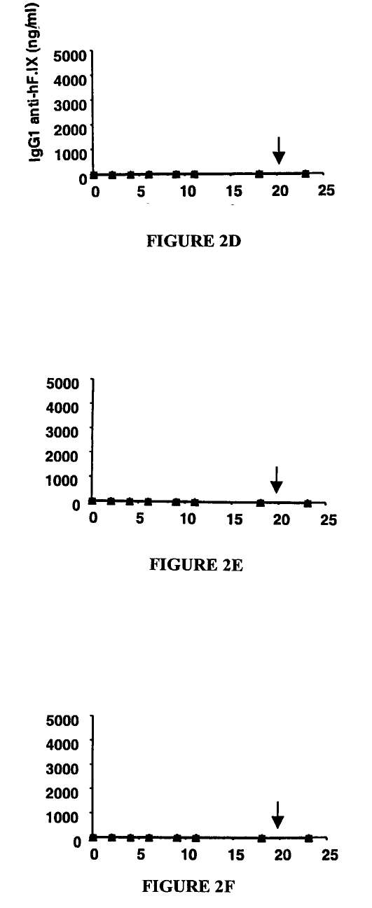

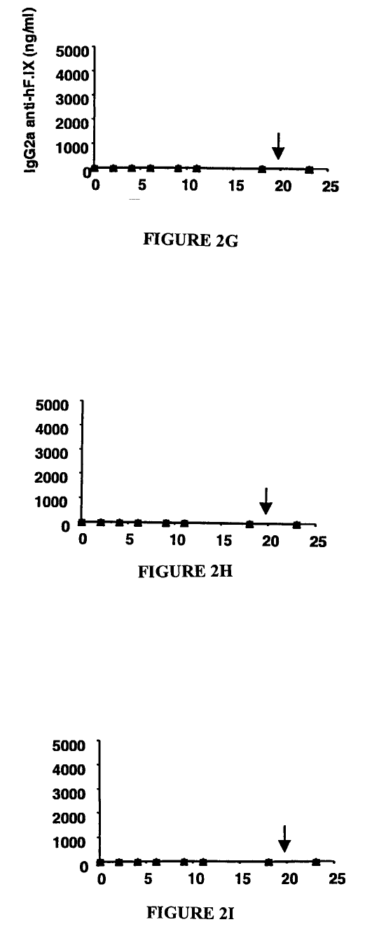

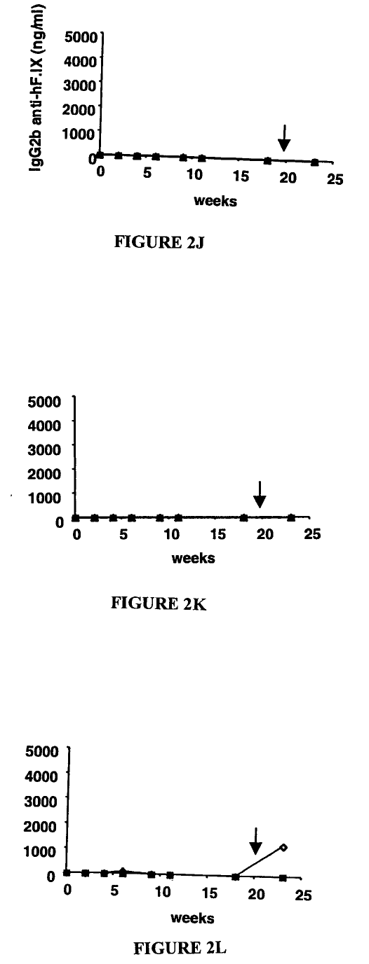

In other experiments with the AAV-EFlα-hF.IX vector, we have also observed a late IgG2b response in BALB/c mice (data not shown). Following gene transfer with the AAV-ApoE/hAAT-hF.IX vector, all mice displayed sustained expression without any detectable anti-hF.IX formation (FIG. 2). Levels of expression were substantially higher with this vector compared to the EFlα promoter. For both vectors, C57BL/6 gave the highest expression levels followed by BALB/c, followed by C3H mice. None of the injected mice had circulating IgA, IgE, or IgG3 anti-hF.IX (data not shown).

### EXAMPLE 4.

Sustained expression of F.IX is associated with induction of immune tolerance

Since mice of different strains showed sustained expression of the hF.IX antigen and failed to mount a neutralizing anti-hF.IX response following hepatic gene transfer, we sought to investigate the nature of this immunological unresponsiveness to the transgene product. Unresponsiveness of the immune system may be the result of ignorance, e.g. due to lack of efficient antigen-derived peptide presentation following this route of administration. If the murine immune system was simply ignoring the hF.IX antigen, an immune response should occur given the proper immunological challenge. Alternatively, transgene expression may have induced immune tolerance.

Example 4.1 AAV vector induces antigen-specific immune tolerance in mice

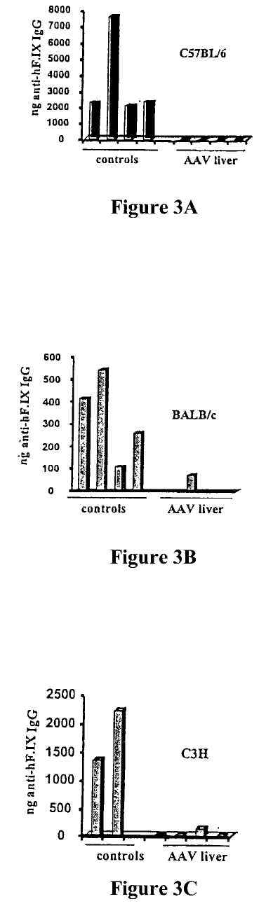

Each of the naïve control mice (four per strain) and the mice that received liver-directed AAV-EFlα-hF.IX gene transfer were challenged with one subcutaneous injection of 2 αg hF.IX protein in cFA. While naïve control mice had high titer anti-hF.IX 14 days after the antigen challenge, 4/4 C57BL/6, 3/4 BALB/c and 4/4 C3H mice continued to express hF.IX without induction of anti-hF.IX IgG (FIG. 3).

Since tolerized mice lacked immune responses after stringent immunological challenge, unresponsiveness cannot be explained by ignorance. Thus, hepatic gene transfer does not simply avoid immune responses, but induces tolerance to the hF.IX transgene product.

Example 4.2 Higher levels of transgene expression favor tolerance.

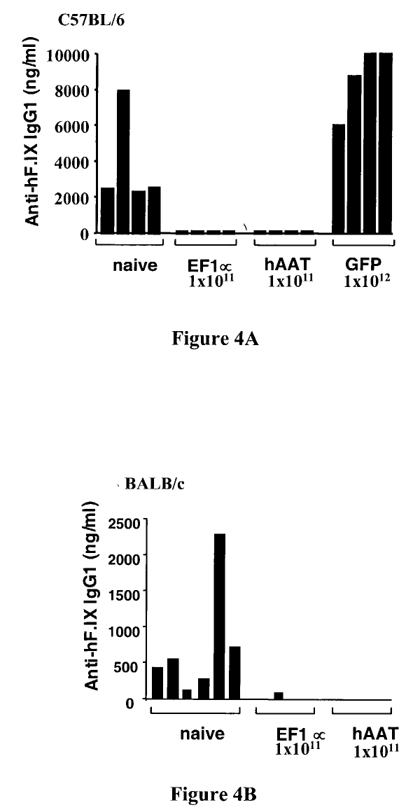

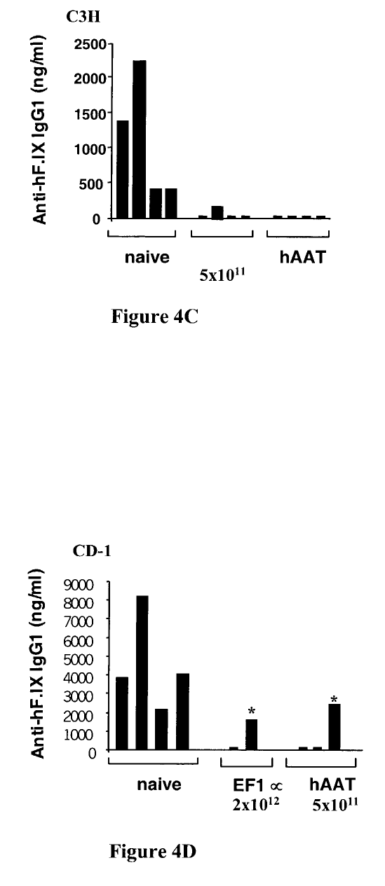

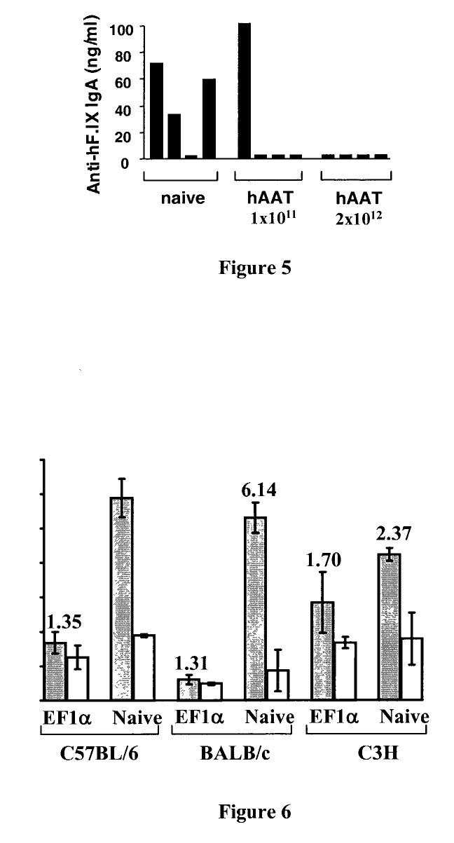

When we performed hepatic gene transfer in the outbred CD-1 strain, 3/4 mice did not have detectable hF.IX plasma levels 2 weeks after injection of lxlO1l AAV-EFlα-hF.IX, and 4/4 mice developed anti-hF.IX by week 4 (FIG. 5A-C). Antibody subclass analyses revealed primarily IgG2a production (indicating a primarily Th1-driven response), and additionally IgG1 and IgG2b anti-hF.IX. After in vitro re-stimulation of lymphocytes (week 10 post-injection) with hF.IX antigen, substantial secretion of IFN-γ and IL-2 was measured confirming activation of Thl helper cells (data not shown). Based on results obtained with C3H mice (see above), we hypothesized that antibody formation could be avoided by an increase in vector dose. Subsequent injection of CD-1 mice with increasing vector doses (4×10¹¹ vg/animal or 2×10¹² vg/animal) confirmed this hypothesis. The mid-dose cohort showed mixed results with mice showing IgG1 or IgG2a anti-hF.IX, or sustained expression without anti-hF.IX. In the high dose cohort, sustained expression was achieved in 4/4 mice (3/4 animals without anti-hF.IX, 1/4 animals with non-neutralizing IgG1). Injection of the more powerful AAV-ApoE/hAAT-hF.IX vector gave sustained sub-therapeutic levels of expression (10-20 ng/ml, lower than in the 3 strains described above) at a dose of 1×10¹¹ vg/animal, while anti-hF.IX formation without detectable expression was observed at lower vector doses. Therapeutic levels of expression were measured in the high dose cohort (5×10¹¹ vg/animal, again 3/4 animals without anti-F.IX, 1/4 animals with non-neutralizing IgG1). Therefore, levels of expression as determined by vector dose and promoter strength rather than tissue-specificity of the promoter determined incidence of antibody formation. Mice treated with 1×10¹¹ AAV-ApoE/hAAT-hF.IX produced low-titer anti-hF.IX after immunological challenge with hF.IX in cFA, whereas mice in the high dose cohorts of either vector generally did not produce anti-hF.IX after. Those mice with IgG1 anti-hF.IX prior to cFA injection, 1/4 in each high dose cohort, continued to express hF.IX, but showed an increase in antibody titer. Note that in contrast to naïve mice, none of the vector treated animals had detectable IgGa after challenge (not shown). In FIG. 4, note that IgG1 anti-F.IX levels are shown as an increase over levels prior to cFA challenge for those mice that had IgG1 anti-hF.IX after gene transfer.

Synthesis of a particular immunoglobulin subclass is caused by activation of a particular subset of T helper cells. Absence of IgG2a and IgG1 production after hepatic gene transfer reflects reduced likelihood of Th1 and Th2-mediated anti-hF.IX formation, respectively. We noticed that naïve CD-1 mice, following a second challenge with hF.IX in adjuvant, produced IgA anti-hF.IX in addition to IgG subclasses (FIG. 5). Mice treated with high vector doses did not produce this immunoglobulin subclass suggesting that there was also a decrease in the potential for Th3-mediated antibody formation (FIG. 5).

Example 4.3 Unresponsiveness to hF.IX antigen is induced on the T helper cell level

Anti-F.IX formation in protein therapy as well as in gene therapy is a T helper cell-dependent process (Fields (2000), Quian et al., Blood (2000) 95:1324, and Reding et al., Advances in Experimental Medicine and Biology (2001) 489:119). In order to investigate whether induction of immune tolerance is reflected in CD4⁺ T helper cell responses, we challenged tolerized mice a second time with hF.IX in incomplete Freund's adjuvant (iFA) and sacrificed the animals 5 days later for in vitro re-stimulation of lymphocytes with hF.IX antigen. As compared to naïve mice challenged in parallel, antigen-specific release of Th2 marker cytokine IL-10 and Th1 cytokines IL-2 and IFN-γ were either undetectable or reduced by 1-2 logs in tolerized mice in all four strains of mice tested (Table 1). Secretion of the potentially immune suppressive cytokine TGF-β was measured in lymphocyte cultures from C57BL/6 and BALB/c mice which had been tolerized with the AAV-ApoE/hAAT-hF.IX vector (Table 1). In agreement with cytokine release data, lymphocytes from AAV-EFlα-hF.IX transduced mice showed no (C57BL/6 and BALB/c) or limited (C3H) proliferation following in vivo challenge with hF.IX/iFA and in vitro re-stimulation with hF.IX antigen, whereas the identical experiment resulted in a proliferative response to hF.IX after immunization of naïve mice of these strains (FIG. 6). In order to assess the possibility that unresponsiveness of T helper cells in hepatic tolerized mice to hF.IX was due to T cell anergy, we attempted to induce cytokine release and proliferation by addition of murine IL-2 to lymphocyte media (see Materials and Methods, experiment performed with C57BL/6 and BALB/c mice). However, unresponsiveness could not be reverted by in vitro incubation with IL-2 (data not shown).

TABLE 1
Cytokine release by pooled lymphocytes (n = 3/strain) following in vitro re-stimulation with hF.IX
protein. Naive or AAV-EFPα-hF.IX injected or AAV-ApoE/hAAT-hF.IX injected mice (portal infusion of
1 × 10^{11-2 × 10}¹² vg/animal as indicated) were boosted twice with hF.IX formulated
in adjuvant (1.5-2.5 months after gene transfer for vector treated mice for the first challenge with hF.IX/cFA
and 1 month later with hF.IX/iFA) and sacrificed on day 5 after the second boost. Total pooled splenocytes, portal
and inguinal lymph node cells (n = 3/strain) were cultured in the presence or absence (mock) of hF.IX antigen
(10 μg/ml media) The limit of detection for the TGF-β ELISA was 50 ng/ml.

| | STRAIN | | | | | | | | | | | |
|---|---|---|---|---|---|---|---|---|---|---|---|---|
| | C57BL/6 | | | BALB/c | | | C3H | | | CD-1 | | |
| AAV vector | Naive | EF1α | hAAT | Naive | EF1α | hAAT | Naive | EF1α | hAAT | Naive | EF1α | hAAT |
| Dose([vg]/ | — | 1 × 10¹¹ | 1 × 10¹¹ | — | 1 × 10¹¹ | 1 × 10¹¹ | — | 5 × 10¹¹ | 1 × 10¹¹ | — | 2 × 10¹² | 5 × 10¹¹ |
| mouse) | | | | | | | | | | | | |
| IFN-γ | 442 | 0 | 42 | 2615 | 0 | 0 | 17421 | 78 | 22 | 2156 | 0 | 0 |
| (pg/ml) | | | | | | | | | | | | |
| IL-2 (pg/ml) | 393 | 5 | 0 | 1114 | 0 | 13 | 1114 | 130 | 2 | 2527 | 0 | 0 |
| IL-10 | 8 | 0 | 0 | 51 | 0 | 0 | 525 | 19 | 0 | 2153 | 0 | 0 |
| (pg/ml) | | | | | | | | | | | | |
| TGF-β | <50 | <50 | 194 | <50 | <50 | 50 | <50 | <50 | <50 | <50 | <50 | <50 |
| (pg/ml) | | | | | | | | | | | | |

Example 4.4 Tolerance induction is antigen-specific

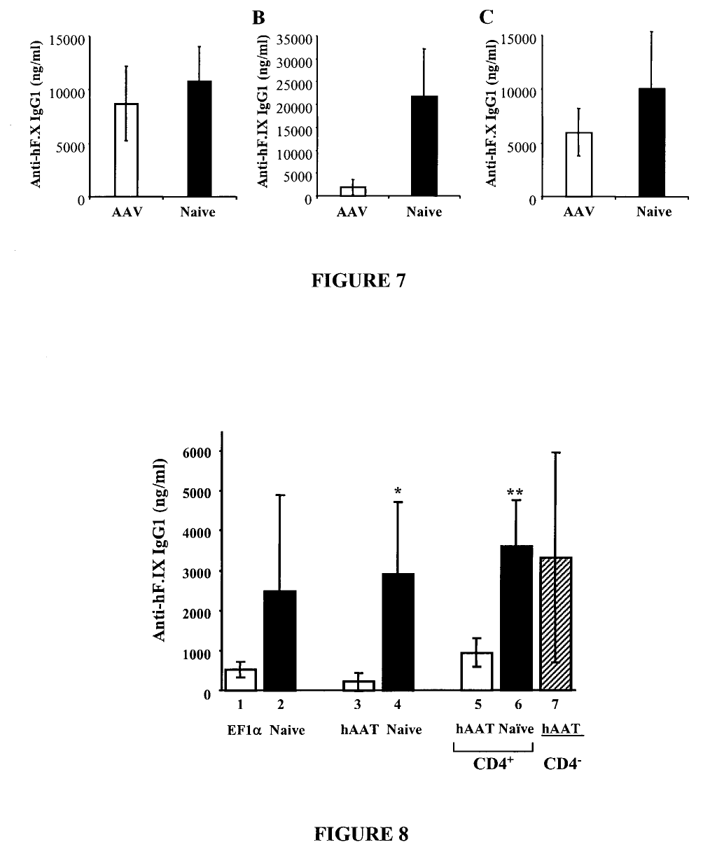

To test antigen-specificity of tolerance induction, we challenged C57BL/6 mice that had received AAV-hF.IX vector by subcutaneous injection of the closely related serine protease hF.X formulated in cFA. These mice formed anti-hF.X at titers indistinguishable from naïve control mice (FIG. 7). Next, we performed subcutanous injections of a mix of hF.IX and hF.X (5 μg each per mouse) in cFA in C57BL/6 mice that had received hepatic AAV-hF.IX gene transfer. As shown in FIG. 7 B, C, naïve mice formed high titer anti-hF.IX and anti-hF.X as expected, while mice with hepatocyte-derived hF.IX expression only formed high titer anti-hF.X, illustrating antigen-specificity of tolerance induction.

Example 4.5 Requirements for tolerance induction.

In order to test requirements for tolerance induction by hepatic F.IX gene transfer, we performed injections of AAV-EFlα-hF.IX vector in several knock out strains (C57BL/6 genetic background) deficient in molecules that effect immune function. CD4⁺ and CD8⁺ T cells express a T cell receptor composed of α and β subunits. Previous work has shown that mice depleted of T cells expressing the rarer γδ-T cell receptor are more difficult to orally tolerize to antigens ( Ke et al., J. Immunol. (1995) 158:3610). Literature on oral tolerance also describes induction of regulatory CD8⁺ T cells secreting TGF-β cytokine (Chen et al., J. Immunol. (1995) 155:910 and Miller et al., J. Exp. Med. (1991) 174:791). However, both γδ-T cell receptor-deficient mice and CD8⁺ T cell-deficient mice showed sustained expression of hF.IX without anti-hF.IX formation following hepatic gene transfer (Table 2). Immunological unresponsiveness was upheld after challenge with hF.IX/cFA (Table 2).

TABLE 2

| | Prior to hF.IX/cFA | | After hF.IX/cFA | |
|---|---|---|---|---|
| | challenge | | challenge | |
| | Sustained | | Sustained | |
| | systemic | | systemic | |
| | hF.IX | Anti-hF.IX | hF.IX | Anti-hF.IX |
| Strain | expression | formation | expression | formation |
| γδ-TCR deficient | Yes | No | Yes | No |
| CD8⁺ T cell | Yes | No | Yes | No |
| deficient | | | | |
| IL-4 deficient | Yes | No | Yes | No |
| Fas deficient | Yes | No | 4/6 mice | Yes (6/6 mice, |
| | | | only | IgG1) |

Liver-directed gene transfer with AAV-EF1α-hF.IX vector (2 × 10¹¹ vg/animal) in knock out mice deficient in γδ-T cell receptor (TCR), CD8^{+ T cells, IL-4 cytokine expression, or Fas. Plasma levels of hF.IX were 100-300 ng/ml in mice with sustained expression. Animals received immunological challenge by subcutaneous injection of hF.IX in cFA 1-2 months after vector administration.}

In previous studies on muscle-directed gene transfer with AAV-F.IX vector, we found a predominantly Th2-driven anti-F.IX response. Since results documented above show a predominant Th1 response in the context of low levels of hF.IX expression in liver-directed gene transfer, one could hypothesize that transduced liver is prone to produce a Th1 response, but at higher expression levels this Th1 response is suppressed by regulatory Th2 cells. To test this interpretation, we transduced IL-4-deficient mice, which by definition cannot produce Th2 cells. These mice also showed sustained expression without evidence for anti-hF.IX (Table 2). In particular, no IgG2a was detected, even after hF.IX/cFA challenge, indicating that tolerance induction cannot be explained by suppression of an imminent Thl response by Th2 cells.

In order to evaluate a potential requirement for apoptotic cell death mediated by the Fas-Fas ligand pathway, we performed hepatic gene transfer in Fas-deficient C57BL/6 mice. Similar to normal C57BL/6 controls, these mice did not develop anti-hF.IX during the first month after vector administration. At this time point, mice were challenged with hF.IX/cFA. Subsequently, Fas-deficient mice produced IgGI anti-hF.IX (6/6) mice within 2 weeks after challenge, while normal controls did not. However, this immune response was neutralizing in only 2/6 mice, while 4/6 Fas-deficient animals continued to show circulating hF.IX levels. Mice with a neutralizing response had high titer anti-hF.IX, while the other 4 animals developed only low titer anti-hF.IX.

Example 4.6 Tolerance is not broken by challenge with adenoviral or muscle-directed gene transfer.

Although challenge with antigen formulated in cFA is a stringent test for tolerance, we wanted to further test whether expression of the antigen in other tissues or from other viral vectors with the ability to activate different subsets of T cells can break tolerance. For example, IM injection of an AAV-hF.IX results in anti-hF.IX formation primarily due to activation of the Th2 subset of T helper cells, while IM administration of an adenoviral vector with hF.IX expression driven by the CMV enhancer promoter additionally results in substantial activation of Th1 cells and in an inflammatory cellular immune response in the transduced muscle tissue. In contrast to AAV, adenoviral vectors efficiently transduce dendritic cells in vivo resulting in MHC class I (in addition of MCH class II) presentation of transgene product-derived peptides. Similar to our previously published results with the AAV-2 serotype, IM administration of ad-hF.IX vector or AAV-CMV-hF.IX (serotype 1) in naïve C57BL/6 mice (4×10¹⁰ viral particles/mouse injected into 2 IM sites in the hindlimbs) elicited an IgGi anti-hF.IX response. In sharp contrast to these results, increase of hF.IX expression without anti-hF.IX formation was measured after IM injection of these vectors in mice that had received hepatic AAV2-EFlα-hF.IX gene transfer 2 months earlier. Use of the AAV-1 serotype in muscle-directed gene transfer results in ˜20-fold higher levels of F.IX transgene expression than with AAV-2. Interestingly, anti-F.IX formation in naïve C57BL/6 mice following AAV-1 gene transfer to skeletal muscle was measured in the presence of high levels of hF.IX in the circulation. Additional experiments will be necessary to assess whether these antibodies interfere with the functional activity of the hF.IX antigen in coagulation. Similar to results described above, adenoviral gene transfer to skeletal muscle also did not elicit anti-hF.IX formation in hepatic tolerized mice, while naïve mice showed IgG1 and IgG2a responses as expected. There was a substantial increase in hF.IX expression after IM injection of ad-hF.IX in tolerized mice. However, expression levels returned to those measured prior to the IM injections (i.e. to liver-derived expression from the AAV vector) within 8 weeks. The transient nature of the added muscle-derived expression may be explained by cellular immune responses against adenovirally transduced muscle fibers. Nonetheless mice not tolerant to hF.IX antigen generally show much more rapid loss of ad-transduced fibers (2-3 weeks) which is likely the result of immune responses against both adenoviral proteins and the transgene product.

In the past, several laboratories have documented transient expression of hF.IX in BALB/c due to a neutralizing anti-hF.IX response after systemic (tail vein) administration of the adenoviral vector described above. While this result was reproducible in naïve BALB/c mice, BALB/c mice that were expressing low amounts of hF.IX after hepatic gene transfer with the AAV2-EFlα-hF.IX showed only a transient low-titer IgG1 anti-hF.IX response after tail vein injection of ad-hF.IX, and furthermore displayed sustained high levels of hF.IX expression as a result of adenoviral gene transfer. As opposed to naïve mice, no IgG2a or IgG2b anti-hF.IX was detected after adenoviral gene. These results indicate limited Th2- and complete absence of Thl-mediated immune responses against the transgene product after systemic challenge with ad-hF.IX vector.

### EXAMPLE 5.

Sustained expression of mouse F.IX (mF.IX) in hemophilia B mice.

Example 5.1 Treatment of hemophilia B mice with large F.IX gene deletion.

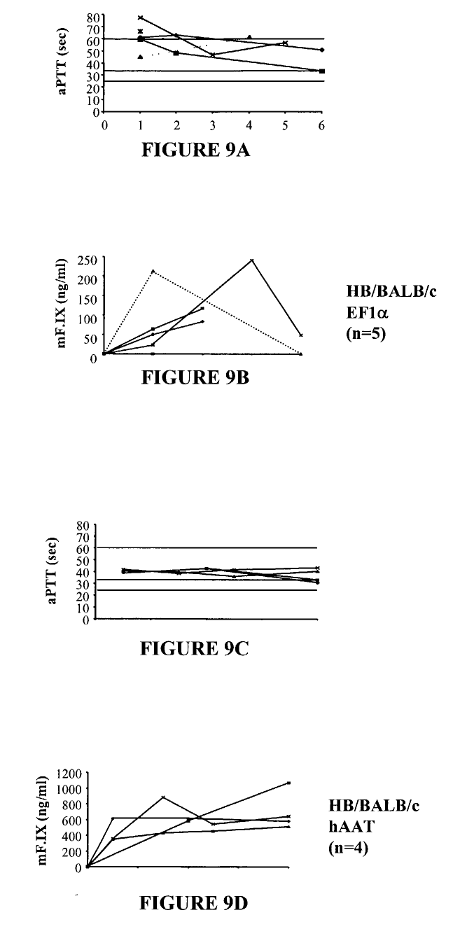

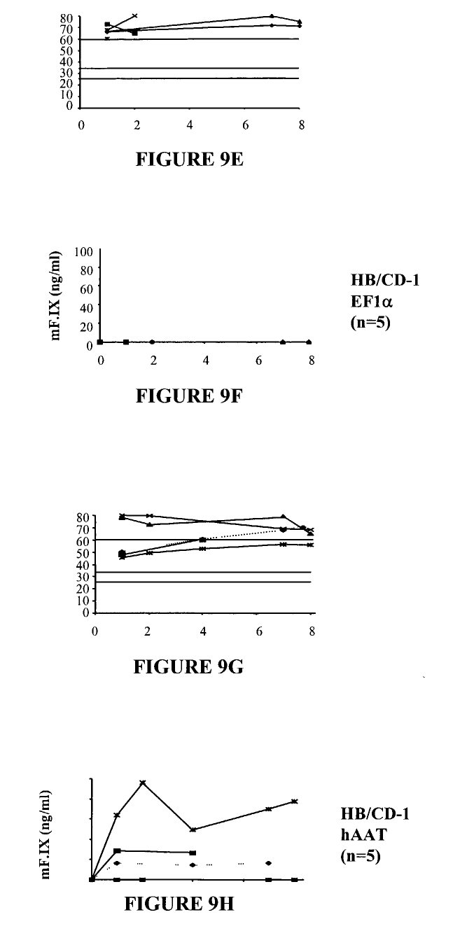

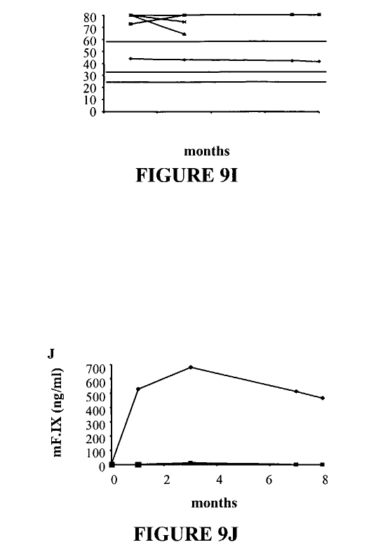

In order to test tolerance induction to F.IX by hepatic gene transfer in animal models of hemophilia B, we bred F.IX knock out mice (without endogenous F.IX protein or mRNA due to a targeted deletion of the promoter region and exons 1-3 of the F.IX gene), onto 3 different genetic backgrounds. These included BALB/c, C3H and CD-1. We did not include C57BL/6 mice, since sustained expression of various F.IX transgenes from different viral vectors following intravenous or portal infusion of the vector in this strain is well documented in the literature (Snyder et al., Nat. Med. (1999) 5:64 and Wang et al., Proc. Natl. Acad. Sci USA (1999) 96:3906). As shown in FIG. 9, expression of mF.IX was obtained after hepatic gene transfer in 4/5 BALB/c mice treated with the AAV-EFlα-mF.IX and in 4/4 BALB/c mice treated with the AAV-ApoE/hAAT-mF.IX vector (3×10¹¹ vg of either vector per mouse for all hemophilic mice injected). In CD-1 mice, expression was achieved in 3/5 mice injected with AAV-ApoE/hAAT-mF.IX vector, while none of the AAV-EFlα-mF.IX injected mice showed mF.IX expression in the circulation (0/5).

Mice with mF.IX expression generally had no detectable anti-mF.IX with two exceptions. While anti-mF.IX antibodies were absent in ¾ hemophilia B mice on a BALB/c background that were administered AAV-ApoE/hAAT-mF.IX vector, and non-neutralizing antibodies were detected in only ¼ mice. These mice continued to express mF.IX for more than 4 months with substantial correction of the activated partial thromboplastin time (aPPT).Additionally, there was one mouse with transient expression at 1 month followed by inhibitor formation at later time points (FIG. 9 A,B, dotted line). This BALB/c hemophilia B mouse synthesized TGF-β dependent IgA and IgG2b anti-mF.IX at 1 month, which was not neutralizing to mF.IX expression or partial correction of coagulation (Table 3 and FIG. 9 A,B). However, this immune deviation toward a Th3-type response shifted to a Th1 response with neutralizing IgG2a by month 4 (Table 3 and FIG. 9 A,B). In contrast, expression was sustained in all other mice that had levels at the 1-month time point. Those mice that did not express mF.IX in the circulation had developed inhibitory anti-mF.IX (Table 4). Inhibitory anti-mF.IX included IgG1 and IgG2a subclasses (data not shown).

TABLE 3

| | | 1 month | 4 months |
|---|---|---|---|
| mF.IX expression | ELISA (ng/ml) | 213 | 0 |
| | aPTT (sec)* | 44.9 | 61.4 |
| Anti-mF.IX | IgG1 (ng/ml) | 0 | 0 |
| | IgG2a (ng/ml) | 0 | 305 |
| | IgG2b (ng/ml) | 150 | 0 |
| | IgA (ng/ml) | 835 | 0 |

Transient immune deviation toward Th3 response in a hemophilia B BALB/c mouse after liver-directed gene transfer with AAV-EFIα-mF.IX vector (3 × 10¹¹ vg). *The aPTT values of untreated Hemophilia B mice were consistently >60 sec, while normal mice had aPTT values of 25-35 sec.

TABLE 4

| | | Before challenge | | After challenge with mF.IX/cFA | |
|---|---|---|---|---|---|
| | | Sustained mF.IX | Inhibitor | Sustained mF.IX | Inhibitor |
| Strain: | Vector | expression | formation | expression | formation |
| BALB/c | EF1α | 3/5 | 2/5 | 1/1 | 0/1 |
| | | (50-250 ng/ml) | 2-5 B.U.) | (50 ng/ml) | |
| | hAAT | 4/4 | 0/4 | 4/4 | 0/4 |
| | | (300-1000 ng/ml) | | (400-1000 ng/ml) | |
| CD-1 | EF1α | 0/5 | 5/5 | — | — |
| | | | (6-10 B.U.) | | |
| | hAAT | 3/5 | 2/5 | 1/2 | 1/2 |
| | | (30-250 ng/ml) | (2-6 B.U.) | (200 ng/ml) | (7 B.U.) |
| C3H | hAAT | 1/5 | 4/5 | 1/1 | 0/1 |
| | | (500 ng/ml) | (5-11 B.U.) | (500 ng/ml) | |
| Immunity vs. tolerance in hemophilia B mice of different strain backgrounds that received hepatic gene transfer with AAV-EF1α-mF.IX (EF1α) or AAV-ApoE/hAAT-mF.IX (hAAT) vector (3 × 10¹¹ vg/mouse). For those mice that had developed inhibitory anti-mF.IX, the range of antibody titer is given in Bethesda Units (B.U.). A total of 8 mice with sustained mF.IX expression was chosen for subcutaneous administration of mF.IX (10 μg) in cFA | | | | | |
| #2-4 months after vector administration. | | | | | |

Some of those mice that showed sustained mF.IX expression were challenged by subcutanous administration of mF.IX in cFA (2-4 months after vector administration) and assayed 1.5 months later for transgene expression and inhibitor formation. Of the 8 mice challenged, 7/8 mice continued to express mF.IX (without evidence for inhibitor formation) at a level identical to that prior to challenge, while 1 hemophilia B CD-1 mouse developed an inhibitor after challenge (Table 4). This mouse had the lowest level of transgene expression (˜30 ng/ml), whereas all mice expressing >50 ng/ml did not form inhibitors after challenge. While the success rate of tolerance induction for these vector/strain combinations was as predicted from experiments with hF.IX in hemostatically normal mice (see above, i.e. higher levels of expression such as in BALB/c mice vs. CD-1 mice or with the ApoE/hAAT vs. the EFlα promoter gave a higher success rate), C3H mice gave a much lower rate of success than predicted (only 1/5 mice injected with AAV-ApoE/hAAT-mF.IX, FIG. 9 I,J).

### EXAMPLE 6.

Example 6.1 Adoptive T cell transfer

C57BL/6 mice (naïve or 1.5 months after hepatic gene transfer with AAV vector, n=4-5 animals per experimental group) were sacrificed, and splenocytes isolated 31. Total splenocytes were pooled and injected via the tail vein (5×10⁷ cells in PBS/recipient naïve C57BL/6 mouse, n=4-5 per experimental group). Recipient mice were subcutaneously injected with hF.IX in cFA as described above 24 hrs after adoptive splenocyte transfer. Anti-hF.IX formation was measured 14 days after immunization. Alternatively, CD4⁺ T cells were purified from pooled splenocytes by magnetic cell sorting (MACS) using a column for positive selection with anti-murine CD4 (Miltenyi Biotech, Auburn, Calif.) according to manufacturer's instructions. FACS analysis showed >75% purity of CD4⁺ T cells with <1% CD8⁺ T cells and <3% B cells. Splenocytes depleted for CD4⁺ T cells contained ˜6% CD8⁺ T cells, <1% CD4⁺ T cells, and ˜70% B cells (as determined with B220 monoclonal anti-CD45R, Pharmingen). CD4⁺ T cells (1×10⁷/recipient mouse) or CD4⁺ T cell-depleted splenocytes (5×10⁷/recipient) were adoptively transferred to naïve C57BL/6 mice followed by hF.IX/cFA challenge 24 hrs later as outlined above.

Example 6.2 Evidencefor CD4⁺ regulatory T cells

If tolerance induction involves regulatory or suppressor lymphocytes, we should be able to transfer unresponsiveness to the hF.IX antigen from tolerized animals to naïve mice of the same strain. To address this question, we adoptively transferred pooled splenocytes from C57BL/6 mice that had received hepatic gene transfer or from naïve C57BL/6 mice (controls) to naïve C57BL/6 mice (5×10⁷ of total splenocytes were injected into the tail vein). Mice were challenged by subcutaneous injection of hF.IX in cFA 24 hrs after receiving splenocytes, and plasma samples analyzed for anti-hF.IX 2 weeks after the challenge. As compared to controls, mice that had received cells from vector-treated animals produced on average 4-8-fold lower IgGI anti-hF.IX levels (FIG. 8). This result was similar for splenocyte transfer from AAV-EFlα-hF.IX and AAV-ApoE/hAAT-hF.IX treated mice. When purified CD4⁺ T cells were transferred (1×10⁷ cells/animal), an identical result was obtained, whereas CD4⁺ T cell-depleted splenocytes (5×10⁷/animal) failed to transfer unresponsiveness (FIG. 8).

### EXAMPLE 7.

Sustained F.IX expression following AAV(ApoE)4/hAAT-cF.IX administration in hemophilia B dogs

The experimental animals (Brad, Beech and Semillon) used in this study were Lhasa Apso-Basenji cross dogs from the Hemophilia B colony housed at the Scott-Ritchey Research Center, Auburn University. These dogs were males with severe hemophilia B caused by a 5-base-pair deletion and a C to T transition in the F.IX gene that results in an early stop codon and unstable FIX transcript. Mauser et al., Blood 1996, 88:3451. One of the dogs (Beech) treated with the AAV vector also had pyruvate kinase deficiency an erythrocyte metabolism disorder. Whitney et al., Exp Hematol. 1991, 22:866. Additionally, a hemophilia B dog with a F.IX missense mutation (E34) of the UNC-Chapel Hill colony was treated. Evans et al., PROC. NAT'L ACAD. SCI. USA 1989, 86:10095. All animals were housed in USDA approved facilities, and the experimental protocol was approved by the Institutional Animal Care and Concern Committee.

The animals were premedicated with diazepam (5 mg) and/or butorphanol (5 mg) and atropine (0.6 mg) prior to anesthetic induction with isoflurane. A midline laparotomy was performed, a mesenteric vein was then isolated and a 20-gauge catheter inserted and tied off with stay sutures. The AAV-(ApoE)4/hAAT-cF.IX vector was administered by slow bolus infusion and the catheter flushed with 5-10 ml heparinized saline before removal and ligation of the mesenteric vein. The abdomen was closed using standard surgical procedures. Butorphanol was administered PRN to provide post-operative analgesia. The dogs were prophylactically administered 90 ml of plasma immediately prior to surgery and 45 ml 8-12 hrs later. Abnormal reactions or toxicity were not noted following vector administration based on clinical examination and routine clinical pathology tests.

Two animals from the Auburn dog colony (Brad and Semillon) and one animal from the UNC-Chapel Hill colony (E34) received vector at a dose of ˜1×10¹² vg/kg (Table 1). Pre-treatment, none of these animals had detectable circulating cF.IX antigen or cF.IX activity owing to a F.IX null mutation (early stop codon associated with unstable F.IX mRNA, Auburn dogs) or a F.IX missense mutation (UNC dog). Evans et al., 1989; Mauser et al., 1996.

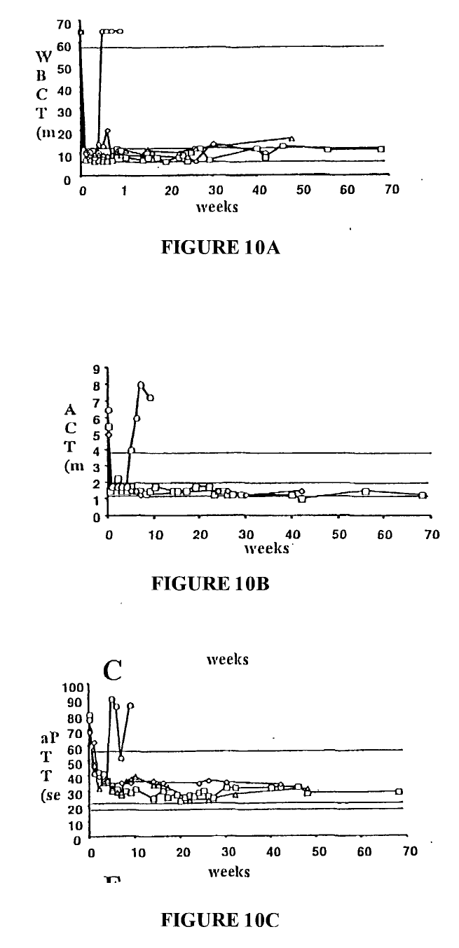

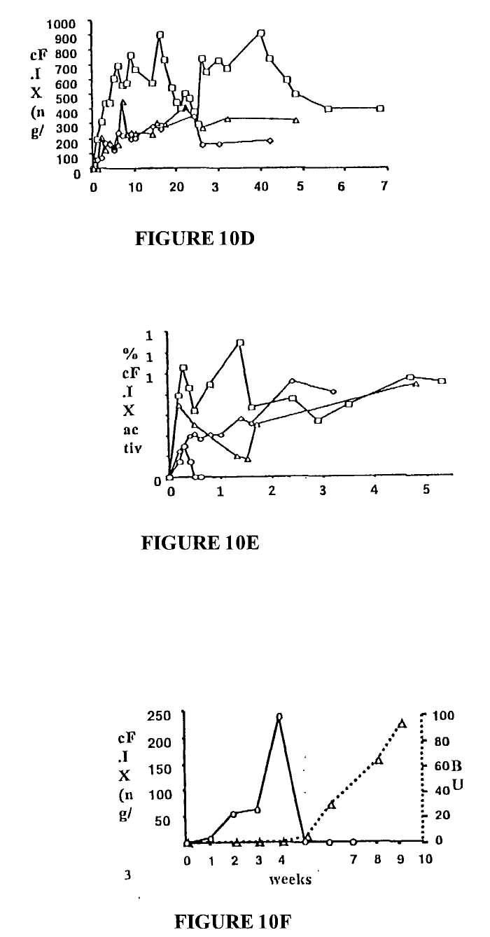

Brad, the first dog treated, also received a total of 180 cc of plasma on day 0 before, during and following surgical laparotomy and vector administration, and 45 cc daily for the next four days (All other animals received only ˜135 cc of plasma prior and just after surgery.) By day 14, or 10 days after the last plasma infusion, the activated clotting time (ACT) in Brad was 1.5 minutes (normal range is 1-2 minutes), compared to 5.5 minutes the day prior to vector administration. The ACT has remained in the normal range for >20 months following vector administration (FIG. 10 B). During the same period of time, the whole blood clotting time (WBCT) was within the normal range (12.1±2.6 minutes vs. >60 minutes pre-treatment), and a PTT (activated partial thromboplastin time) values (29.4±3.6 seconds) were significantly shortened from pre-treatment times of 79.9 seconds (FIG. 10 A,C). Canine F.IX antigen was undetectable prior to vector administration but had increased to 317 ng/ml by week 2 and peaked at 907 ng/ml on week 16 (FIG. 10 D). Antigen levels of 590±150 ng/ml have persisted for the duration of the study. Likewise, cF.IX activity of 8.6±2.1% of a canine plasma pool has also persisted for the >20 month observation period (FIG. 10 E and Table 5). The dog also had a normal cuticle bleed time post-treatment (data not shown).

TABLE 5

| | | | Total | | | | | | cFIX |
|---|---|---|---|---|---|---|---|---|---|
| | Age | | Dose | Dose/kg | | WBCT | aPTT | cF.IX | activity |
| Animal | (months) | Weight³ | (vg) | (vg/kg) | PK | (min) | (sec) | (ng/ml) | (%) |
| Brad¹ | 9 | 10.2 kg | 1.25 × 10¹³ | 1.2 × 10¹² | No | 12 ± 2.5 | 29.5 ± 3.5 | 590 ± 150 | 8.5 ± 2 |
| Semillon | 5.5 | 6.0 kg | 9.7 × 10¹² | 1.6 × 10¹² | No | 13.5 ± 4 | 35.5 ± 2 | 220 ± 65 | 5 ± 2.5 |
| Beech¹ | 12 | 10.5 kg | 3.6 × 10¹³ | 3.4 × 10¹² | Yes | ≧10 | ≧36.2 | ≦2560 | ≦3 |
| E34² | 5 | 12.3 kg | 9.6 × 10¹² | 8 × 10¹¹ | No | 11 ± 2.5 | 32 ± 4.5 | 262 ± 92 | 5 ± 2.5 |

The other two dogs (E34 and Semillon) also showed sustained, complete or nearly complete correction of the WBCT and/or ACT (not measured in E34) and substantial correction of the aPTT from >60 sec pretreatment to ˜32-35 sec (see Table 5 and FIG. 10 A-C). The cF.IX antigen levels averaged 220±65 ng/ml for Semillon and 262±92 ng/ml for E34 (FIG. 10 D). FIX activity averaged 4.9±2.6% of normal canine plasma for Semillon and 5±2.5% for E34 (FIG. 10 E and Table 5). Expression was sustained in both animals for >15 months in Semillon and >14 months in E34.

A third null mutation dog, Beech, was injected with 3.4×10¹² vg/kg (˜3-times higher vector dose, see Table 1). WBCT and ACT values were within the normal range following gene transfer (weeks 2-4), but returned to baseline by week 5 (FIG. 10 A,B). The aPTT results were consistent with these observations, showing decreasing values through week 4 (without ever achieving a normal value), but returning to a greater than pre-treatment value of 90.4 sec by week 5 (FIG. 10 C). The cF.IX antigen level rose to >2 mg/ml by week 4 but had dropped to 13 ng/ml by week 5, and was undetectable by week 6 (FIG. 10 F). F.IX activity showed a similar pattern, rising from 0% to 1.3% by week 2, peaking at 3.0% on week 3 and returning to 0% by week 5 (FIG. 10 E). As shown below, loss of systemic cF.IX expression was due to formation of an inhibitory anti-cF.IX that first emerged at week 5. The discrepancy between cF.IX antigen levels measured by ELISA and cF.IX activity levels in Beech likely are due to the presence of an anti-phospholipid antibody in this animal (vide infra) as determined by RVVT assay and described before for a different animal of this colony. Herzog et al., 2001. At 11 weeks after vector administration, Beech developed a fatal intra-abdominal bleed, which, due to a lack of canine bypass reagents such as factor VIIa, could not be treated.

| | | | | | | | | | cFIX |
|---|---|---|---|---|---|---|---|---|---|
| | Age³ | | Total Dose | Dose/kg | | | aPTT | cF.IX | activity |
| Animal | (months) | Weight³ | (vg) | (vg/kg) | PK | WBCT (min) | (sec) | (ng/ml) | (%) |
| Brad¹ | 9 | 10.2 kg | 1.25 × 10¹³ | 1.2 × 10¹² | No | 12 ± 2.5 | 29.5 ± 3.5 | 590 ± 150 | 8.5 ± 2 |
| Semillon¹ | 5.5 | 6.0 kg | 9.7 × 10¹² | 1.6 × 10¹² | No | 13.5 ± 4 | 35.5 ± 2 | 220 ± 65 | 5 ± 2.5 |
| Beech¹ | 12 | 10.5 kg | 3.6 × 10¹³ | 3.4 × 10¹² | Yes | ≧10 | ≧36.2 | ≦2560 | ≦3 |
| E34² | 5 | 12.3 kg | 9.6 × 10¹² | 8 × 10¹¹ | No | 11 ± 2.5 | 32 ± 4.5 | 262 ± 92 | 5 ± 2.5 |

### EXAMPLE 8.

F.IX, coagulation, and antibody assays in dogs

Blood samples were drawn from hemophilia B dogs as described. Herzog et al., 2001. The whole blood clotting time (WBCT), activated clotting time (ACT), activated partial thromboplastin time (aPTT) of plasma samples, and F.IX activity levels were measured as previous reported. Herzog et al., 1999; Herzog et al., 2001. Canine F.IX antigen levels in plasma samples were determined by ELISA. Herzog et al., 1999; Herzog et al., 2001. Anti-cF.IX was demonstrated by immunocapture assay specific to canine IgG1, IgG2, IgM, and IgA immunoglobulins, by Western blot, or by Bethesda assay as described previously. Herzog et al., 2001; Fields et al., 2001. One Bethesda Unit (BU) represents inhibition of normal F.IX activity by 50%. Anti-phospholipid was detected by dilute Russel's Viper Venom Time. Neutralizing antibodies (NAB) against AAV2 vector particles were measured by inhibition of in vitro LacZ transduction as described. Herzog et al., 2001. The treated animals did not have a pre-existing NAB titer, but all developed NAB to AAV2 post-vector administration.

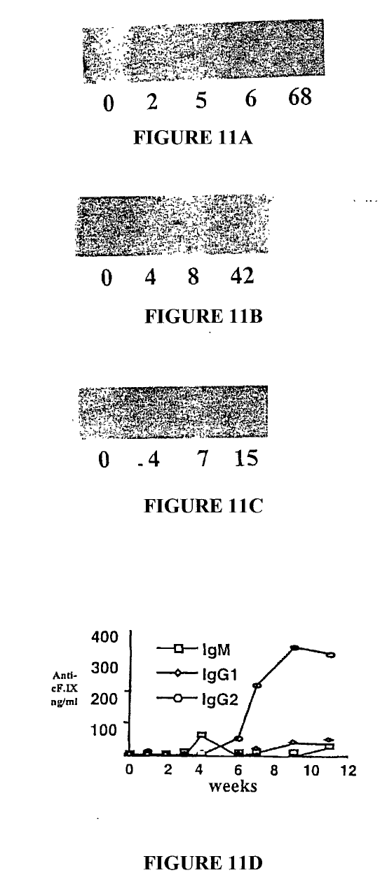

Beech, the dog with transient F.IX expression, developed F.IX-specific antibodies concomitant with the loss of F.IX antigen and activity. The Bethesda titer increased from 0 (pre-treatment through week 4) to 4.0 B.U. on week 5 with a subsequently rising titer (FIG. 10 E). Anti-cF.IX IgG was undetectable in serum from week 0 through week 4, but was demonstrated in week 5 and subsequently by Western blot. Immnunocapture assay showed synthesis of IgM at week 4, followed by high titer IgG2 anti-cF.IX at week 5 and low titer IgG1 at week 9 (FIG. 11 D). Brad, Semillon, and E34, the dogs with sustained F.IX expression, had no evidence for anti-c.F.IX by Western blot, immunocapture assay, or Bethesda assay at any time point tested (FIG. 11 A-C). IgA anti-cF.IX was not detected in any of the treated animals and no animals had anti-cF.IX pre-treatment.

### EXAMPLE 9.

DNA analysis

Total genomic DNA was isolated from canine liver or spleen tissue using the Easy DNA kit from Invitrogen. Vector-specific sequences were detected by Southern blot hybridization using a 0.9-kb probe specific to the human α1-antitrypsin promoter and intron sequences in the AAV vector.

What is claimed is:

1. A gene therapy method for a human subject, comprising (A) providing a composition comprised of a vector and a polynucleotide encoding a therapeutic polypeptide to which said subject is immunologically competent and (B) administering said composition to the subject, such that (i) said therapeutic polypeptide is expressed selectively in hepatocytes of the subject and thereafter (ii) said subject fails to generate a medically-significant immune response to the expressed therapeutic polypeptide.

2. The method of claim 1, wherein said composition further comprises a pharmaceutically suitable excipient.

3. The method of claim 1, wherein said composition is administered intravenously.

4. The method of claim 3, wherein said intravenous administration is effected through the group consisting of the portal vein, mesenteric vein and hepatic artery of said subject.

5. The method of claim 1, wherein said polynucleotide is operably linked to a liver-specific promoter.

6. The method of claim 5, wherein said liver-specific promoter is a human α1-antitrypsin promoter.

7. The method of claim 1, wherein said polynucleotide is operably linked to ubiquitous promoter.

8. The method of claim 1, wherein said vector can be selected from the group consisting of a plasmid, an adenovirus vector, an adeno-associated virus vector, herpes simplex virus vector, lentivirus vector and retrovirus vector.

9. The method of claim 8, wherein said vector is an adeno-associated virus vector.

10. The method of claim 1, wherein said therapeutic polypeptide modulates the blood clotting or coagulation cascade.

11. The method of claim 10, wherein said subject suffers from hemophilia.

12. The method of claim 11, wherein said subject suffers from hemophilia B and said therapeutic polypeptide that modulates the blood clotting or coagulation cascade is factor IX.

13. The method of claim 1, exclusive of using an immunomodulator.

14. The method of claim 12, exclusive of using an immunomodulator.

15. The method of claim 1, wherein said composition is administered before said subject has exhibited immune intolerance to said therapeutic polypeptide.

16. The method of claim 10, wherein said composition is administered before said subject has exhibited immune intolerance to functional factor IX.
